(19) United States
(12) Patent Application Publication
(10) Pub. No.: US 2023/0226085 A1
(43) Pub. Date: Jul. 20, 2023

(54) SURFACTANT COMPOSITIONS AND METHODS FOR EMULSIFYING CANNABINOID EXTRACTS AS A NANO-EMULSION MATERIAL

(71) Applicant: Advanced NanoEmulsions Inc, Calgary (CA)

(72) Inventor: Steven Paul Hansen, Nanaimo (CA)

(21) Appl. No.: 18/155,634

(22) Filed: Jan. 17, 2023

Related U.S. Application Data
(63) Continuation-in-part of application No. 16/542,079, filed on Aug. 15, 2019, now abandoned.

(51) Int. Cl. A61K 31/00; A61K 47/14; A61K 47/24; A61K 9/107; A61K 47/22; A61K 47/26; A61K 47/10; A61K 47/44

(52) U.S. Cl. CPC A61K 31/658; A61K 47/14; A61K 47/24; A61K 9/1075; A61K 47/22; A61K 47/26; A61K 47/10; A61K 47/44

(57) ABSTRACT

An embodiment of a CBD or other cannabinoid or terpene or lipid-soluble Nano-Emulsion material and the process comprises a formulation comprising at least a lecithin or mixed lecithin, one or more carrier oils, and a Vitamin E TPGS from a sunflower version and a soy version or Polysorbate 80, 60 or 65. Among either version, the versions may further comprise mixed tocopherols and still further comprise at least one of; an LCT oil, an olive oil, and coconut oil. Any one of the versions may further comprise at least one of; sodium benzoate, potassium sorbate, and sorbic acid, and even yet further comprise purified water. Some embodiments may comprise a Vitamin E acetate or beeswax.

### CROSS-REFERENCES TO RELATED APPLICATIONS

This application is a continuation-in-part and claims benefit of U.S. patent application Ser. No. 16/542,079 filed Aug. 15, 2019, the specifications of which are incorporated herein in their entirety by reference.

### FIELD OF THE INVENTION

This disclosure relates generally to surfactant compositions and methods to create a nano-emulsion for a cannabinoid extract so as to be miscible in water, thereby creating a product to be used as a component in the production of ingestible goods.

### BACKGROUND OF THE INVENTION

Cannabis extracts, as an oil-based component, are generally not water soluble or miscible with water. There are currently technologies that exist that render cannabinoids water soluble, but such technologies alter the cannabinoid molecules by functionalizing with groups such as sugars or phosphate esters. Creating new and untested altered cannabinoid molecules is technology that may be of interest to other third parties, but the present disclosure is directed to maintaining cannabis in its native form. Water miscible cannabis can be created using natural cannabis extracts in conjunction with emulsifiers derived from natural sources and with GRAS ratings, and that is the strategy disclosed herein.

There are several ways in which to render cannabis extracts miscible with water. Broadly, those consist of low energy processes and high energy processes. Low energy processes require more emulsifiers and are typically not infinitely dilutable in water. Further, emulsions made with low energy processes (so-called SEDDS or SNEDDS or SNED emulsions) are not stable with respect to dilution ie., particle size decreases with increasing dilution. High energy processes require a minimum quantity of emulsifiers, and when combined with the correct surfactant technology are infinitely dilutable. Particle sizes do not change with dilution. High energy processes include technologies such as sonication and high-pressure homogenization. Those technologies are well-known. The present disclosure of novel surfactant technology is designed to work in conjunction with these high energy emulsification processes.

The prior art does not provide surfactant formulations for water-miscible extracts having all of the following properties: 1. Infinitely dilutable in water (e.g., particle size does not change upon dilution), 2. Kinetically stable over long time periods, 3. GRAS surfactant technologies, 4. translucent concentrates, 5. Availability in soy and non-soy versions (GMO free and non-GMO), 6. Temperature stable from about 0° C. to about 85° C., 7. Suitable for use with any high-energy emulsification process, 8. Stable storage in both plastic and glass containers when diluted or not diluted, 9. Stable over a pH range from about 1 to about 13, 10. Compatible with a variety of products and cannabinoid delivery systems, including gummies (both gelatin and agar/pectin), transdermal patches, beverages (encompassing fruit juices with natural and artificial flavors, water, energy drinks and sport recovery drinks), foods Gello, baked goods), 11. Workable in ratios from about 5:1 surfactant:active ingredient to about 2:1 surfactant:active ingredient, 12. Compatible with a wide variety of carrier oils, including MCT, olive, grapeseed, coconut, LCT, almond, apricot kernel, avocado, canola, hemp, castor, jojoba, palm kernel, rosehip seed, borage seed, camellia seed, cranberry seed, hazelnut, macadamia nut, peanut, pomegranate, sesame, sunflower, watermelon seed, and the like, and 13. Tasteless (G2 embodiment) and odorless.

### BRIEF SUMMARY OF THE INVENTION

It is an objective of the present invention to provide compositions and methods that allow for cannabinoid nanoemulsion compositions, as specified in the independent claims. Embodiments of the invention are given in the dependent claims. Embodiments of the present invention can be freely combined with each other if they are not mutually exclusive.

This disclosure teaches a cannabinoid nanoemulsion material comprising at least one formulation selected from a G1 family, wherein any such version comprises at least a lecithin, a carrier oil, such as MCT oil, and a polysorbate, such as a polysorbate 80; a single component sunflower version, a single component soy version, a two-component sunflower version Part A and Part B, and a two- component soy version Part A and Part B. Among the G1 family versions, a polysorbate-80 may be replaced by one or more of; a polysorbate 60, and a polysorbate 65. Any of the G1 family versions may further comprise at least one of; an LCT oil, an olive oil, and coconut oil, and any of the single component family versions may further comprise at least one of; a yellow beeswax, sodium benzoate, potassium sorbate, sorbic acid, and vitamin E acetate. The G1 family versions may further comprise purified water.

This disclosure teaches a cannabinoid nanoemulsion material comprising at least one formulation selected from a G2 family, wherein any such family version comprises at least a lecithin, a carrier oil such as MCT oil, and a Vitamin E TPGS from a sunflower version and a soy version. Among the G2 family versions, the G2 family versions may further comprise mixed tocopherols and still further comprise at least one of; an LCT oil, an olive oil, and coconut oil. Any one of the G2 family versions may further comprise at least one of; sodium benzoate, potassium sorbate, and sorbic acid, and even yet further comprise purified water.

In some embodiments, the present invention features a cannabinoid nanoemulsion composition. The cannabinoid nanoemulsion composition may comprise a lecithin (e.g., a water insoluble lecithin), a carrier oil, a water soluble emulsifier, preservatives, water, and a cannabinoid extract. In some embodiments, the cannabinoid nanoemulsion composition may comprise 5-40 wt % lecithin (e.g., a water insoluble lecithin), 5-50 wt % carrier oil, 10-50 wt % water soluble emulsifier, 0-0.2 wt % preservatives, 0-2 wt % water, and 1-30 wt % cannabinoid extract. In other embodiments, the cannabinoid nanoemulsion composition may comprise 5-40 wt % water insoluble lecithin, 5-50 wt % carrier oil, 10-50 wt % water soluble emulsifier, 0-0.2 wt % preservatives, 0-2 wt % water, and 1-30 wt % cannabinoid extract. In some embodiments, the lecithins (e.g., the water insoluble lecithin) comprises phosphatidylinositol, phosphatidylethanolamine, phosphatidylcholine, phosphatidylserine, or a combination thereof. In some embodiments, the cannabinoid nanoemulsion compositions described herein are kinetically stable. In some embodiments, kinetically stable means dilution of the cannabinoid nanoemulsion composition does not affect a range of particle sizes in the composition (See FIG. 5). In some embodiments, the composition is infinitely dilutable in water.

In some embodiments, the lecithins comprise a single lecithin or multiple lecithins. In some embodiments, the carrier oil is long chain triglyceride (LCT) oil, medium chain triglyceride (MCT) oil, coconut oil, olive oil, or a combination thereof. In some embodiments, the water soluble emulsifier is vitamin E TPGS, Polysorbate 80, Polysorbate 60, Polysorbate 65, or a combination thereof. In some embodiments, the Vitamin E TPGS is derived from a sunflower source or soy source.

In some embodiments, the composition may further comprise a long chain triglyceride (LCT) oil, an olive oil, a coconut oil, or a combination thereof. In some embodiments, the composition may further comprise yellow beeswax, vitamin E acetate, glycerine, or a combination thereof. In some embodiments, the composition may further comprise mixed tocopherols. In some embodiments, the composition may further comprise preservatives. In some embodiments, the preservative comprises sodium benzoate, potassium sorbate, sorbic acid, or a combination thereof.

One of the unique and inventive technical features of the present invention is the use of water insoluble lecithins that contain relatively high amounts of phosphatidylinositol or phosphatidylethanolamine. In such embodiments, inositol and ethanolamine head groups interact with serine and choline head groups to form stable micellar and liposomal structures upon the input of energy from either a high pressure homogenizer or a sonicator. Without wishing to limit the invention to any theory or mechanism, it is believed that the technical feature of the present invention advantageously provides for molecular packing that stabilizes a cannabinoid extract in a micellar or liposomal structure. Additionally, these micellar and liposomal structures are relatively rigid and impenetrable and are not disrupted by dilution. Furthermore, it is believed that the technical feature of the present invention advantageously provides for a composition that is kinetically stable and infinitely dilutable in water. None of the presently known prior references or work has the unique, inventive technical feature of the present invention.

Furthermore, the prior references teach away from the present invention. For example, Schwarz et al. (US20200022386A1) utilize SNED (self-nano-emulsifying delivery) systems to create the cannabinoid nanoemulsion material described therein. However, the SNED system described by Schwarz cannot achieve what can be achieved by the sonication and homogenization methods claimed by the present invention.

First, there is a clear distinction between the methodologies. The SNED systems, as used in Schwarz, require a higher quantity of emulsifiers and co-emulsifiers as well as solvents and co-solvents because these systems must form emulsions without any input energy. Thus, Schwarz teaches away from the present invention, which creates a formulation that requires input energy. The sonication and high-pressure homogenization used in the present invention add substantial amounts of energy into cannabinoid nanoemulsion material to disrupt and disassemble larger particles. Again, the SNED systems described in Schwarz require the formulations to spontaneously self-assemble into small particles in a low-energy process.

Additionally, in some embodiments, lecithins comprising phosphatidylinositol and phosphatidylethanolamine are incompatible with SNEDDS formulations

Secondly, the SNED systems are thermodynamically stable, in a low energy state and in chemical equilibrium with the environment. As such, putting energy into the formulations described in Schwarz will not cause them to enter a lower energy state. Furthermore, Schwarz teaches that adding the nanoemulsion material to cold or room temperature water or beverages results in turbidity or the production of large visible oil droplets. For example, paragraph [0102] states, “. . . mixing . . . the preparation . . . at 5° C. . . . in one step (ratio 1:100) lead[s] to the formation of visible cloudiness, turbidity, and visible aggregates/particles . . . . ” This requires that Schwarz use a two-step dilution process, where thermal energy is initially added to the system while hydrating it, and then the nanoemulsion material is added to water or the intended beverage (paragraph [0125]). Thus, Schwarz teaches away from using a one-step dilution process.

Contrarily, the formulation of the present invention uses high-energy methods to form micelles and liposomes and is kinetically stable but NOT thermodynamically stable. This means that the particles are not in their lowest energy state with respect to the surrounding environment and are NOT in thermodynamic equilibrium with the environment. Therefore, the cannabinoid nanoemulsion material presently claimed can be added to cold or room temperature water or beverages in one step without disrupting the nanoemulsion particles, producing a translucent and stable dispersion.

Furthermore, the Schwarz formulations, when subjected to high energy processes, still form SNEDDS-type emulsions where particle size is not stable with respect to dilution.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A and 1B shows THC distillate made with self-nanoemulsifying drug delivery systems (SNEDDS) (e.g., with no energy). FIG. 1A shows a 10 to 1 dilution with a 10:1 ratio of SNEDDS emulsifier and THC and FIG. 1B shows a 500 to 1 dilution with a 10:1 ratio of SNEDD emulsifier to THC. The height of bars on the graph indicates the percentage of particles of a given size (% channel), and the integral line represents a sieve analysis, where % passing means the percentage of particles that are smaller than a given size, as read on the x-axis.

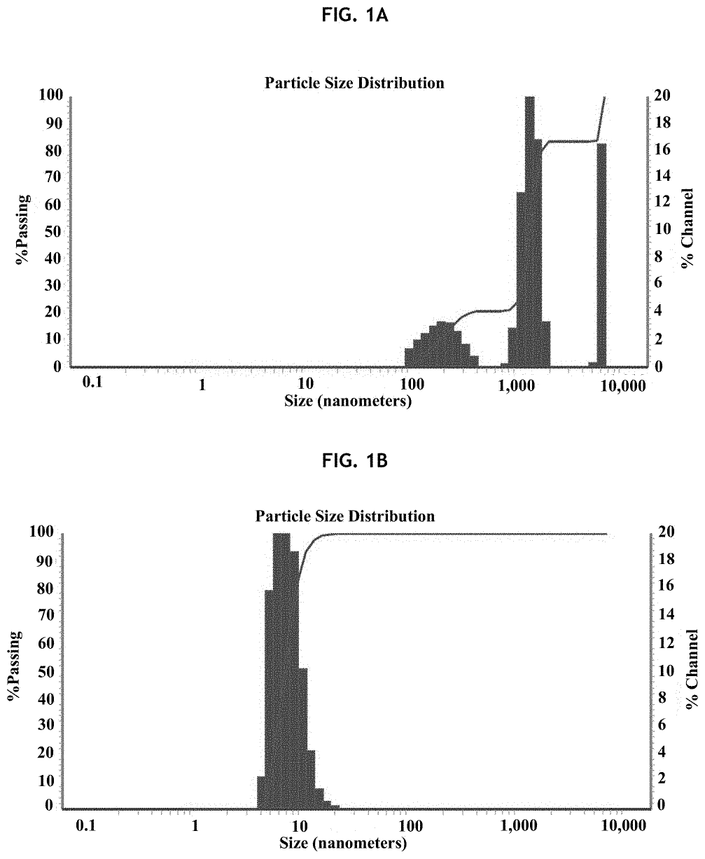

FIGS. 2A and 2B show THC distillate made from the compositions and methods described herein (e.g., with energy). FIG. 2A shows an undiluted composition with a 4:1 ratio of G2 emulsifier and THC. FIG. 2B shows a 500 to 1 dilution with a 4:1 ratio of G2 emulsifier and THC. The height of bars on the graph indicates the percentage of particles of a given size (% channel), and the integral line represents a sieve analysis, where % passing means the percentage of particles that are smaller than a given size, as read on the x-axis.

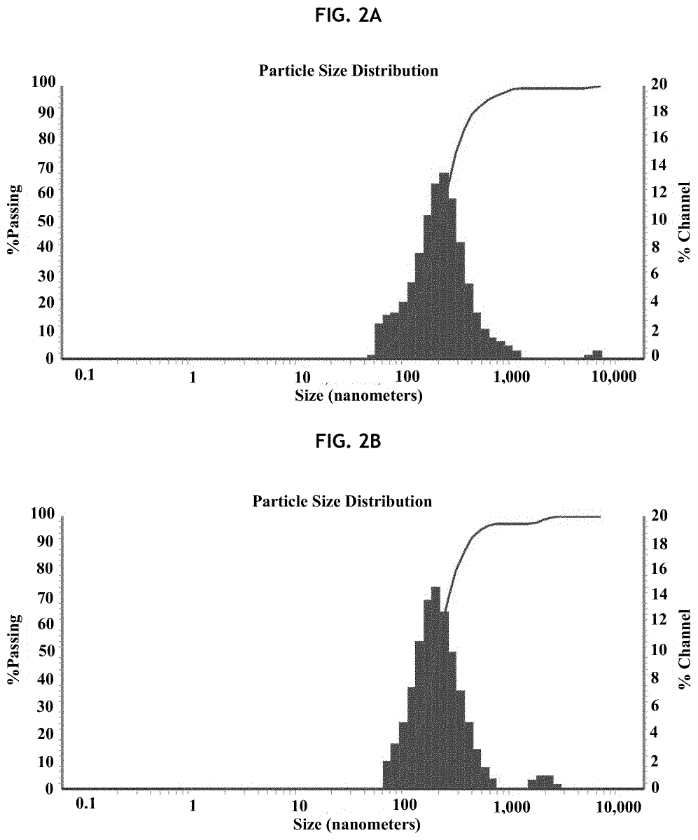

FIGS. 3A and 3B shows a 50 mg/mL CBD isolate made from the compositions and methods described herein (e.g., with energy) with a 4:1 ratio of G2 emulsifier and CBD at a dilution of 300:1 with 2 passes (FIG. 3A) and 3 passes (FIG. 3B). The number of passes indicates the number of times the composition (e.g., emulsion) was passed through a high pressure homogenizer. In some embodiments, increasing the number of passes reduces the particle size. The height of bars on the graph indicates the percentage of particles of a given size (% channel), and the integral line represents a sieve analysis, where % passing means the percentage of particles that are smaller than a given size, as read on the x-axis.

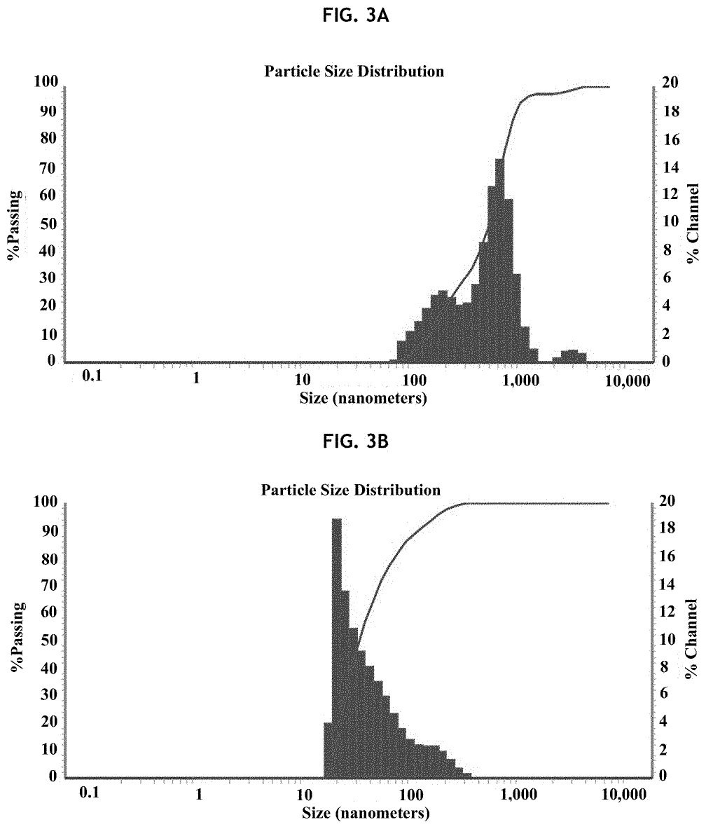

FIGS. 4A and 4B shows a 50 mg/mL CBD isolate made from the compositions and methods described herein (e.g., with energy) with a 2:1 ratio of G2 emulsifier and CBD at a dilution of 200:1 with 2 passes (FIG. 4A) and 3 passes (FIG. 4B). The height of bars on the graph indicate the percentage of particles of a given size (% channel), and the integral line represents a sieve analysis, where % passing means the percentage of particles that are smaller than a given size as read on the x-axis.

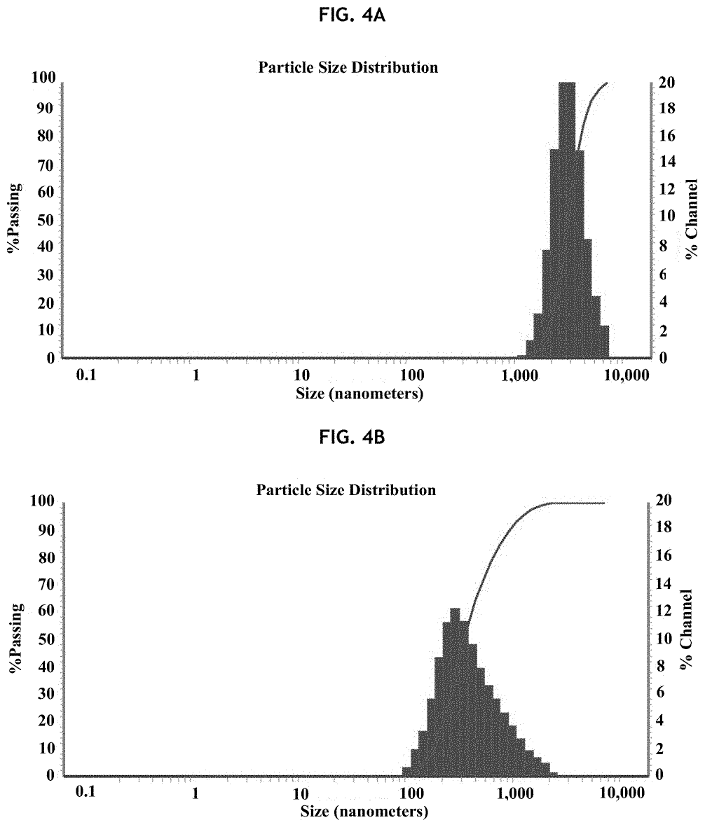

FIG. 5 shows a comparison of the particle sizes of a cannabinoid nanoemulsion composition created using the methods described herein (left side) and using a SN ED system as described in Schwarz (right). The left side images are modified versions of the FIGS. 2A and 2B and the right side images or modified versions of FIGS. 5 and 6 in the Schwarz prior art. The particle size of the compositions described herein does not change as with dilution, whereas the particle size of the compositions described in Schwarz does change as larger particles seen in the top right graph disappear upon a second dilution (bottom, right graph).

### TERMS

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, a “carrier oil” refers to an oil that can dissolve surfactant and active ingredients. In some embodiments, the carrier oil may lower the viscosity of the surfactant and/or active melt to allow easy blending with water. In some embodiments, a surfactant and/or active melt is made when a surfactant (e.g., emulsifier) and an active ingredient are dissolved in each other at a temperature of 40 to 60° C.

As used herein, an “emulsifier” refers to a substance that renders a material (e.g., oil), otherwise insoluble in water, miscible with water. As used herein, “emulsifier” and “surfactant” may be used interchangeably.

As used herein, the term hydrophilic-lipophilic balance (HLB) refers to a numeric measurement of the water solubility of a molecule. In some embodiments, a high HLB molecule refers to a molecule that is water soluble, and a low HLB molecule refers to a molecule that is water insoluble.

As used herein, a “microemulsion” refers to a composition comprising a particle size of about 200 nm to about 2 micrometers.

As used herein, a “nano-emulsion” refers to a composition comprising a particle size of about 10 nm to about 200 nm.

As used herein, “infinitely dilutable in water” refers to a composition (e.g., the compositions described herein) that are stable at any dilution, e.g., the composition will not separate from the bulk solution and the particle size does not change upon dilution.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

Exemplary embodiments herein may be described in terms of functional block components and various processing steps. Such functional blocks may be realized by any number of raw materials, hardware, and/or even software components configured to perform the specified functions and achieve various results. For example, surfactant compositions and methods for emulsifying cannabinoid extracts as a nano-emulsion material may employ various hardware components, e.g., various hardware equipment generally known in the chemical processing arts to process the various formulas, as well as for storage, packaging, distribution equipment and the like, which may carry out a variety of functions. In addition, surfactant compositions and methods for emulsifying cannabinoid extracts as a nano-emulsion material may be practiced in conjunction with other settings, such as for consumer use, commercial use, research, further development of associative materials, and the like. Also, any processes, materials, systems, and/or methods described are merely exemplary applications for the surfactant compositions and methods for emulsifying cannabinoid extracts as a nano-emulsion material. The surfactant compositions and methods for emulsifying cannabinoid extracts as a nano-emulsion material may employ conventional techniques for manufacturing, distributing, marketing, packaging, shipping, and the like.

The present invention features several novel families of surfactants, and these surfactants can effectively surround any cannabinoid, terpene, essential oil, hemp oil, or cannabis extract; and create a stable translucent nano-emulsion with particle sizes in the range of about 10 nm to about 70 nm and concentrations up to about 70 mg/mL. The following formulations and processes may apply to all families of cannabinoids, e.g., CBD, THC, THCV, CBN, and the like. Moreover, the following formulations and processes may apply to various limonenes, pinenes, beta-caryophyllene, and the like.

The present invention features a cannabinoid nanoemulsion composition comprising lecithins (e.g., a water insoluble lecithin), a carrier oil, a water soluble emulsifier, and a cannabinoid extract. In some embodiments, the cannabinoid nanoemulsion composition further comprises preservatives and water. In some embodiments, the lecithins comprise phosphatidylinositol, phosphatidylethanolamine, phosphatidylcholine, phosphatidylserine, or a combination thereof.

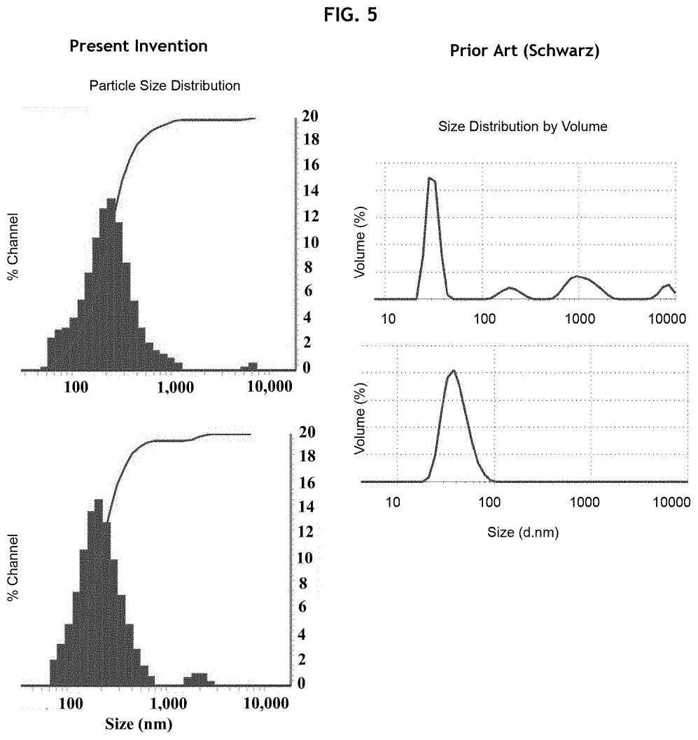

In some embodiments, the cannabinoid nanoemulsion composition may comprise 5-40 wt % lecithin (e.g., a water insoluble lecithin), 5-50 wt % carrier oil, 10-50 wt % water soluble emulsifier, 0-0.2 wt % preservatives, 0-2 wt % water, and 1-30 wt % cannabinoid extract. In other embodiments, the cannabinoid nanoemulsion composition may comprise 5-40 wt % water insoluble lecithin (e.g., phosphatidylinositol and phosphatidylethanolamine), 5-50 wt % carrier oil, 10-50 wt % water soluble emulsifier, 0-0.2 wt % preservatives, 0-2 wt % water, and 1-30 wt % cannabinoid extract. In some embodiments, the water insoluble lecithins comprise phosphatidylinositol, phosphatidylethanolamine, phosphatidylcholine, phosphatidylserine, or a combination thereof. In some embodiments, the cannabinoid nanoemulsion compositions described herein are kinetically stable. In some embodiments, kinetically stable means dilution of the cannabinoid nanoemulsion composition does not affect a range of particle sizes in the composition (See FIG. 5).

In some embodiments, the range of particle sizes in the composition may statistically vary by about 5%. In some embodiments, the range of particle sizes in the composition may statistically vary by about 10%. In some embodiments, the range of particle sizes in the composition may statistically vary by about 15%. In some embodiments, the range of particle sizes in the composition may statistically vary by about 20%. In some embodiments, the range of particle sizes in the composition may statistically vary by about 5-20%, or about 5-15%, or about 5-10%, or about 10-20%, or about 10-15%, or about 15-20%.

Non-limiting examples of lecithins (e.g., water insoluble lecithin) used in compositions described herein include but are not limited to phosphatidylcholine, phosphatidylethanolamine, phosphatidylinositol, phosphatidylserine, phosphatidic acid, or a combination thereof. In some embodiments, the lecithins (e.g., water insoluble lecithin) comprise single lecithin or multiple lecithins. In some embodiments, lecithins (e.g., water insoluble lecithin) comprise phosphatidylinositol, phosphatidylethanolamine, or a combination thereof.

In some embodiments, the carrier oil is a long chain triglyceride (LCT) oil, a medium chain triglyceride (MCT) oil, a coconut oil, an olive oil, or a combination thereof. In some embodiments, the water soluble emulsifier is vitamin E TPGS, Polysorbate 80, Polysorbate 60, Polysorbate 65, or a combination thereof. In some embodiments, the Vitamin E TPGS is derived from a sunflower source or soy source. In some embodiments, the composition further comprises a preservative, wherein the preservative comprises sodium benzoate, potassium sorbate, sorbic acid, or a combination thereof.

In some embodiments, cannabinoid nanoemulsion composition further comprises a long chain triglyceride (LCT) oil, an olive oil, a coconut oil, or a combination thereof. In some embodiments, cannabinoid nanoemulsion composition further comprises yellow beeswax, vitamin E acetate, glycerine, or a combination thereof. In some embodiments, cannabinoid nanoemulsion composition further comprises mixed tocopherols.

The cannabinoid nanoemulsion composition may be infinitely dilutable in water.

### Surfactant Type

In some embodiments, G1 surfactant families, G2 surfactant families, or a combination thereof may be used in compositions described herein.

For example, G1 surfactant families are generally sold and used as two separate components that are combined at the time of use (although a one part G1 is also described herein). Component B comprises polysorbate 80 and water-soluble preservatives, and component A comprises lecithins, carrier oils, and oil-soluble preservatives. Additionally, the G2 surfactant families comprise Vitamin E TPGS, aka Tocopherols, instead of polysorbate 80. Like polysorbate 80, Vitamin E TPGS is synthetic, but it operates as a source of vitamin E. In a non-synthetic embodiment, part or all of the Vitamin E TPGS may be replaced with mixed tocopherols. Mixed tocopherols consist of 4 different vitamin E isomers and have not been chemically modified.

In some embodiments, polysorbate 80 comprises a concentration of about 20 mg/mL. In some embodiments, polysorbate 80 comprises a concentration of about 15 mg/mL. In some embodiments, polysorbate 80 comprises a concentration of about 10 mg/mL. In some embodiments, polysorbate 80 comprises a concentration of about 5 mg/mL.

In some embodiments, component A and component B are used in about equal proportions. In other embodiments, component A is used at a higher proportion than component B. In some embodiments, component A is used at a lower proportion than component B.

G1 and G2 surfactant families may be further broken down into those that contain soy-derived ingredients and those that contain sunflower-derived ingredients. Both lecithin and Vitamin E TPGS may be derived from soy or sunflower sources. Sunflower sources are considered to be preferred in most formulations, as many users are allergic to soy. Soy is also GMO (Genetically Modified Organism), whereas sunflower is not.

Various representative embodiments may be applied to any method or system to surround any cannabinoid, terpene, essential oil, hemp oil, or cannabis extract. Nano-emulsion materials and processes are as follows.

### Ingredients

Carrier Oils

Non-limiting examples of carrier oils may include but are not limited to medium-chain triglycerides (MCT) oils, coconut oil, long chain triglycerides (LCT) oil, olive oil, or a combination thereof. Other oils may be used as carrier oils in accordance with the present invention.

In some embodiments, MCT oils may be derived from coconut sources. MCT oil comprises C6 to C12 triglycerides and is a healthy oil readily absorbed by the body and requires limited low energy to digest. In some embodiments, MCT oil may be an excellent solvent for lecithin and vitamin TGGS and an excellent carrier oil for cannabinoids and terpenes.

Coconut oil comprises C8 to C12 triglycerides, with C12 being the most prominent. In some embodiments, coconut oil may be a superior solvent for lecithin and vitamin E TPGS and an excellent carrier oil for cannabinoids and terpenes.

LCT oils comprise C18 and higher triglycerides, and preferably glyceryl monolinoleate is used. In some embodiments, LCTs may increase bioavailability. Other LCTs are available, and those skilled in the art would employ such others.

Olive Oil comprises primarily oleic acid (C18) esters. In some embodiments, olive oil may be rich in LCT oil, which is more bioavailable than MCT oils and less expensive than pure LCT oil.

Like those listed above, other carrier oils comprising complex mixtures of triglycerides may be used. In some embodiments, other carrier oils may work best in combination with the aforementioned carrier oils; however, other carrier oils may also operate with other carrier oils.

Emulsifiers

Non-limiting examples of emulsifiers (e.g., water soluble emulsifiers) include but are not limited to lecithin, Vitamin E TPGS, Polysorbate 80, Polysorbate 60, Polysorbate 65, or a combination thereof.

Lecithin. There are many grades of lecithin, most of which are unsuitable for use owing to high impurity content. The most suitable grades for use are those in which 100% of the constituents are soluble in ethanol. In some embodiments, lecithins deceived herein may be derived from many sources (e.g., soy and sunflower). In some embodiments, lecithins may comprise choline, linoleic acid, linolenic acid, and the phosphatides phosphatidylcholine, phosphatidylethanolamine, phosphatidylinositol, phosphatidic acid, and various vitamins and minerals. Higher grades of lecithins may comprise more phosphatidylcholine. Lecithin acts as a low HLB emulsifier.

Vitamin E TPGS is a synthetic emulsifier comprising vitamin E, succinic acid, and polyethylene glycol. In some embodiments, vitamin E may be isolated from soy or sunflower sources. In some embodiments, the emulsifiers (e.g., vitamin E TGGS) described herein are not bitter and, when combined with lecithin and the correct carrier oils, make an emulsifier that contributes a minimum of flavor. Along with being an excellent emulsifier, Vitamin E TPGS is a source of vitamin E and serves as an antioxidant. Vitamin E TPGS is a high HLB emulsifier.

As used herein, a “correct carrier oil” refers to any oil that is able to dissolve the lecithins and active ingredients (e.g., cannabinoids) described herein. polysorbate 80 is a synthetic emulsifier made of ethoxylated sorbitan reacted with lauric acid. It is part of the Tween family of emulsifiers. It is very bitter but inexpensive relative to vitamin E TPGS. polysorbate 80 is a high HLB emulsifier. Although polysorbate 80 is the preferred synthetic emulsifier disclosed herein, those skilled in the art will appreciate that other Polysorbates may be employed, such as Polysorbate 60 and/or Polysorbate 65.

Preservatives and Antioxidants

Sodium Benzoate is a water-phase preservative having bacteriostatic and fungistatic properties.

Potassium Sorbate is a water-phase preservative having bacteriostatic and fungistatic properties.

Sorbic Acid is an oil phase preservative having bacteriostatic and fungistatic properties.

Benzyl Alcohol acts in both oil and water phases and has bacteriostatic and fungistatic properties.

Vitamin E TPGS is an antioxidant. It operates as an oxygen scavenger and inhibits the THC to CBN reaction.

Vitamin E acetate is an antioxidant. It functions as a good oxygen scavenger and inhibits the THC to CBN reaction.

Mixed tocopherols are antioxidants. It operates as a good oxygen scavenger and inhibits the THC to CBN reaction.

Compatibilizer

Yellow beeswax allows the fatty earner oils and lecithin to blend with polysorbate without the mixture separating.

Mixed tocopherols allow fatty carrier oils and lecithin to blend with Vitamin E TPGS without the mixture separating.

Freeze/Thaw stability: In some embodiments, glycerin may be added to compositions described herein to increase freeze/thaw stability where required

Processing;

In some embodiments, compositions described herein all comprise lecithin. Lecithin has a very high melting point and must be dissolved into a carrier oil. In an embodiment, a process uses a “chocolate conditioner” that allows for the oil and lecithin to be mixed together at elevated temperatures, from about 50° C. to about 85° C. A chocolate temperer may also be suitable for this purpose. The disclosed equipment significantly reduces dissolution time from 3 days to about 1 day or even less. It will further be appreciated by those skilled in the art that while such chocolate conditioners and/or temperers are preferably employed, other systems that can effectively accomplish the above with similar results may be used. For example, single or multishift mixers with heated bowls and the like may be employed.

Those skilled in the art will appreciate that Vitamin E TPGS is a waxy solid. In formulations that comprise this ingredient, it is added to the chocolate conditioner and melted into the formulation. Sorbic acid may also be dissolved into the warm melt.

Preservatives, including sodium benzoate and potassium sorbate, may be added as a solution in water or as a concentrated blend in a carrier oil or a carrier oil/lecithin blend.

G1 Family

In the single-component G1 version, yellow beeswax is added to the melt. polysorbate 80 and water-soluble preservatives are premixed using, preferably, an overhead mixer. The mixture is then added to the chocolate conditioner and blended with lecithin, oils, and sorbic acid.

In the two-component G1 version (A-B), the polysorbate component is kept separate from the material in the chocolate conditioner. Beeswax is not needed in this formulation. To make an emulsifier, component A (lecithin/oil) is mixed with component B (polysorbate) when the emulsification is carried out.

G2 Family

As with G1, lecithin and sorbic acid are dissolved in the chocolate conditioner. Vitamin E TPGS and/or tocopherols or tocotrienols are added and melted into the material once the lecithin has dissolved. Once homogenous, the mixture is emptied into a food grade mixing container where sodium benzoate and potassium sorbate are added as aqueous solutions with overhead mixing. The resulting product is dispensed while it is at about 40° C. to about 45° C. to avoid solidification; the mixture is moved out of the chocolate mixing machine between about 50° C. to about 65° C.

Formulas

### G1 Single Component

| Soy Version | | Sunflower Version | |
|---|---|---|---|
| | Weight | | Weight |
| | Percentage | | Percentage |
| Ingredients | Range | Ingredients | Range |
| Single or Mixed | 5-40 | Single or Mixed | 5-40 |
| Soy Lecithins | | Sunflower Lecithins | |
| MCT Oil | 10-50 | MCT Oil | 10-50 |
| Polysorbate 80 | 20-80 | Polysorbate 80 | 20-80 |
| Olive Oil | 0-20 | Olive Oil | 0-20 |
| LCT Oil | 0-20 | LCT Oil | 0-20 |
| Coconut Oil | 0-20 | Coconut Oil | 0-20 |
| Purified Water | 0-2 | Purified Water | 0-2 |
| Yellow Beeswax | 0-2 | Yellow Beeswax | 0-2 |
| Sodium Benzoate | 0-0.2 | Sodium Benzoate | 0-0.2 |
| Potassium Sorbate | 0-0.2 | Potassium Sorbate | 0-0.2 |
| Sorbic Acid | 0-0.2 | Sorbic Acid | 0-0.2 |
| Vitamin E Acetate | 0-0.2 | Vitamin E Acetate | 0-0.2 |

### G1 Two Component, Part A

| Soy Version | | Sunflower Version | |
|---|---|---|---|
| | Weight | | Weight |
| | Percentage | | Percentage |
| Ingredients | Range | Ingredients | Range |
| Single or mixed | 10-80 | Single or Mixed | 10-80 |
| Soy Lecithins | | Sunflower Lecithins | |
| MCT Oil | 5-80 | MCT Oil | 5-80 |
| Olive Oil | 0-50 | Olive Oil | 0-50 |
| LCT Oil | 0-20 | LCT Oil | 0-20 |
| Coconut oil | 0-20 | Coconut oil | 0-20 |
| Sorbic Acid | 0-0.4 | Sorbic Acid | 0-0.4 |
| Vitamin E Acetate | 0-0.4 | Vitamin E Acetate | 0-0.4 |

### G1 Two Components, Part B (for either Soy or Sunflower Version, Part A)

| Ingredient | Weight Percentage Range |
|---|---|
| polysorbate 80 | 50-100 |
| Purified water | 0-10 |
| Sodium Benzoate | 0-0.4 |
| Potassium Sorbate | 0-0.4 |

### G2

| Soy Version | | Sunflower Version | |
|---|---|---|---|
| | Weight | | Weight |
| | Percentage | | Percentage |
| Ingredients | Range | Ingredients | Range |
| MCT Oil | 10-50 | MCT Oil | 10-50 |
| Vitamin E TPGS | 10-50 | Vitamin E TPGS | 10-50 |
| Single or Mixed Soy | 5-40 | Single or Mixed | 5-40 |
| Lecithins | | Sunflower Lecithins | |
| Olive Oil | 0-20 | Olive Oil | 0-20 |
| Mixed Tocopherols | 0-20 | Mixed Tocopherols | 0-40 |
| LCT Oil | 0-20 | LCT Oil | 0-20 |
| Coconut Oil | 0-20 | Coconut Oil | 0-20 |
| Purified Water | 0-10 | Purified Water | 0-10 |
| Sodium Benzoate | 0-0.2 | Sodium Benzoate | 0-0.2 |
| Potassium Sorbate | 0-0.2 | Potassium Sorbate | 0-0.2 |
| Sorbic Acid | 0-0.2 | Sorbic Acid | 0-0.2 |

Emulsifying and Post Processing

In a representative embodiment, a surfactant is combined with an active ingredient at a ratio of about 5 parts surfactants to about 1-part cannabinoid extract. Depending on the nature of the active ingredient, the ratio can go as low as 2:1. The mixture is melted together with mixing at a temperature from about 50° C. to about 70° C. When the mixture is homogeneous, heat is removed, and water is added such that the total percentage of cannabinoids (i.e., active ingredient) is about 1% and about 7%. The mixture is stirred with a stir bar, high speed mixer, or overhead mixer until the material is homogenous, about 2 to about 15 minutes. When homogenous, the subsequent mixture is considered a microemulsion and comprises a mixture that is essentially opaque and comprises a white or beige coloration depending on the cannabinoid extract used. Once the microemulsion is formed, it may be sonicated or put through a high-pressure homogenizer. Depending on the probe and power level, sonication times range from about 3 to about 30 minutes. When using a high-pressure homogenizer, the pressure may range from about 15,000 to 45,000 psi. Homogenization time depends on pressure and the internal geometry and configuration of the intensifier chamber. Those skilled in the art will appreciate that any sonication and/or homogenization processes known in the art may be employed, and such process equipment may comprise from 1 to about 15 passes via such equipment so as to achieve particle sizes from about 5 nm to about 1,000 nm.

In some embodiments, when using a high-pressure homogenizer, the pressure may range from about 15,000 to 45,000 psi, or about 15,000 to 40,000 psi, or about 15,000 to 35,000 psi, or about 15,000 to 30,000 psi, or about 15,000 to 25,000 psi, or about 15,000 to 20,000 psi, or about 20,000 to 45,000 psi, or about 20,000 to 40,000 psi, or about 20,000 to 35,000 psi, or about 20,000 to 30,000 psi, or about 20,000 to 25,000 psi, or about 25,000 to 45,000 psi, or about 25,000 to 40,000 psi, or about 25,000 to 35,000 psi, or about 25,000 to 30,000 psi, or about 30,000 to 45,000 psi, or about 30,000 to 40,000 psi, or about 30,000 to 35,000 psi, or about 35,000 to 45,000 psi, or about 35,000 to 40,000 psi, or about 40,000 to 45,000 psi.

Among various embodiments, once the surfactant and active ingredient are entirely homogeneous, water is added with stirring, which creates a microemulsion. After stirring, preferably with a stir bar, high speed mixer, or overhead mixer, for approximately 2-15 minutes, the material may then be put through a high-pressure homogenizer or sonicated. In principle, any technique that imparts a large amount of mechanical energy into the microemulsion may produce a nano-emulsion.

Among various embodiments, the nano-emulsion may be put through a 220 nm filter after emulsification to remove insoluble impurities and any microorganisms that may be present. The product is then sealed in a sterile bottle or other suitable storage container and refrigerated.

In an embodiment, if freeze/thaw stability is required, glycerin may be added at levels of up to about 20%.

In the preceding specification, surfactant compositions and methods for emulsifying cannabinoid extracts as a nano-emulsion material have been described with reference to specific exemplary embodiments. Various modifications and changes may be made, however, without departing from the scope of surfactant compositions and methods for emulsifying cannabinoid extracts as a nano-emulsion material as set forth in the claims. The detailed description is illustrative of representative embodiments rather than restrictive, and modifications are intended to be included within the scope of the various representative embodiments. Accordingly, the scope of the representative embodiments should be determined by the claims and their legal equivalents rather than by merely the examples described.

For example, the steps recited in any method or process claims may be executed in various orders to achieve the desired results and may not be limited to the specific order presented in the claims. Additionally, the components and/or elements recited in any article, material, system, apparatus, and/or device claims may be assembled or otherwise operationally configured in a variety of permutations and are accordingly may not be limited to the specific configuration recited in the claims.

Benefits, other advantages, and solutions to problems have been described above with regard to particular embodiments; however, any benefit, advantage, solution to problem, or any element that may cause any particular benefit, advantage, or solution to occur or to become more pronounced are not to be construed as critical, required, or essential features or components of any or all the claims.

As used herein, the terms “comprise”, “comprises”, “comprising”, “having”, “including”, “includes” “is” or any variation thereof, are intended to reference a non-exclusive inclusion, such that a process, method, article, system, device, material, composition or apparatus that comprises a list of elements does not include only those elements recited, but may also include other elements not expressly listed or inherent to such process, method, article, system, device, material, composition or apparatus. Other combinations and/or modifications of the above-described structures, arrangements, applications, proportions, elements, materials or components used in the practice of the surfactant compositions and methods for emulsifying cannabinoid extracts as a nano-emulsion material, in addition to those not specifically recited, may be varied or otherwise particularly adapted to specific environments, manufacturing specifications, design parameters or other operating requirements without departing from the general principles of the same.

As used herein, the terms “first,” “second,” “third,” ““fourth,” and the like in the description and in the claims, if any, are used for distinguishing between similar elements and not necessarily for describing a particular sequential or chronological order. It is to be understood that the terms so used are interchangeable under appropriate circumstances such that the embodiments of surfactant compositions and methods for emulsifying cannabinoid extracts as a nano-emulsion material described herein are, for example, capable of operation in sequences other than those illustrated or otherwise described herein.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A cannabinoid nanoemulsion composition comprising:
a) 5-40 wt % water insoluble lecithins, wherein the water insoluble lecithins comprise phosphatidylinositol, phosphatidylethanolamine, or a combination thereof,
b) 5-50 wt % carrier oil,
c) 10-50 wt % water soluble emulsifier
d) 0-0.2 wt % preservatives,
e) 0-2 wt % water, and
f) 1-30 wt % cannabinoid extract;
wherein the cannabinoid nanoemulsion composition is kinetically stable; wherein kinetically stables means dilution of the cannabinoid nanoemulsion composition does not affect a range of particle sizes in the composition;

2. The composition of claim 1, wherein the water insoluble lecithins further comprise phosphatidylcholine, phosphatidylserine, or a combination thereof.

3. The composition of claim 1, wherein the water insoluble lecithins comprise a single lecithin or multiple lecithins.

4. The composition of claim 1, wherein the carrier oil is long chain triglyceride (LCT) oil, medium chain triglyceride (MCT) oil, coconut oil, olive oil, or a combination thereof.

5. The composition of claim 1, water soluble emulsifier is vitamin E TPGS, Polysorbate 80, Polysorbate 60, Polysorbate 65, or a combination thereof.

6. The composition of claim 5, wherein the Vitamin E TPGS is derived from a sunflower source or soy source.

7. The composition of claim 1 further comprising a long chain triglyceride (LCT) oil, an olive oil, a coconut oil, or a combination thereof.

8. The composition of claim 1 further comprising yellow beeswax, vitamin E acetate, glycerine, or a combination thereof.

9. The composition of claim 1 further comprising mixed tocopherols.

10. The composition of claim 1 further comprising a preservative, wherein the preservative comprises sodium benzoate, potassium sorbate, sorbic acid, or a combination thereof.

11. The composition of claim 1, wherein the composition is infinitely dilutable in water.
